(19) United States
(12) Patent Application Publication
(10) Pub. No.: US 2019/0175267 A1
(43) Pub. Date: Jun. 13, 2019

(54) DEVICE AND METHOD FOR DETERMINING A LOCAL PROPERTY OF A BIOLOGICAL TISSUE

(71) Applicant: VascoMed GmbH, Binzen (DE)

(72) Inventors: Henning Ebert, Berlin (DE); Jens Rump, Berlin (DE)

(21) Appl. No.: 16/209,211

(22) Filed: Dec. 4, 2018

(30) Foreign Application Priority Data
Dec. 13, 2017 (EP) 17 20 6949.4

(51) Int. Cl. A61B 18/14; G01R 33/28; A61M 25/01; A61B 5/055

(52) U.S. Cl. CPC A61B 18/1492; G01R 33/285; A61B 2018/00577; A61B 5/055; A61M 2025/0166; A61M 25/0127

(57) ABSTRACT

The disclosure relates to an ablation catheter for determining a local property of a biological tissue, said catheter having a flexible shaft, a data processing device, and an NMR sensor, which is arranged at the distal end of the shaft and is connected to the data processing device, wherein the NMR sensor comprises a first sensor element for generating a static magnetic field and a second sensor element for generating a magnetic alternating field, wherein the distal end of the shaft can be arranged adjacently to the point of the tissue to be measured, wherein the data processing device is designed to determine the local property of the tissue at this point on the basis of a signal of the NMR sensor transmitted to the data processing device. The disclosure also relates to a corresponding method.

### CROSS-REFERENCE TO RELATED APPLICATIONS

This patent application claims the benefit of and priority to co-pending European Patent Application No. EP 17206949.4, filed on Dec. 13, 2017 in the European Patent Office, which is hereby incorporated by reference in its entirety.

### TECHNICAL FIELD

In conjunction with the ablation of biological tissue, for example in order to optimize the impulse conduction in the heart (electrophysiology), destruction of nerves (renal denervation) or tumor treatment, the knowledge of tissue properties, for example lesion depth or local thickness of the treated tissue, is of utmost important for the assessment of therapeutic success of the intervention. The present invention therefore concerns a device and a method for determining a local property of a biological tissue.

### BACKGROUND

A known method for non-drug-based, minimally invasive treatment of idiopathic, paroxysmal, persistent or chronic arrhythmias, in particular supraventricular arrhythmias, of the heart is intracardiac ablation. Here, in the case of atrial fibrillation or other arrhythmias, such as atrial flutter, a catheter with an electrode is inserted via the venous blood vessels into the right atrium of the heart and is placed in the left atrium through the cardiac septum. Areas of muscle tissue in the left atrium are then destroyed (ablated) by means of a high-frequency current introduced through the electrode, in such a way that what are known as rotors (rotating stimuli) or ectopic activation sources are remedied and pulmonary veins are isolated, these being deemed to be a cause of arrhythmias. Alternatively, a minimally invasive treatment by means of laser, freezing, heat radiation, microwave energy or particle therapy can be performed analogously.

The success rate of an ablation can be increased if, during the treatment, the efficacy of the lesions induced by the ablation or other minimally invasive procedures can be assessed more reliably. This is possible with current methods only to a limited extent. In many cases, a second AF ablation is therefore also necessary after a first AF ablation treatment (AF=atrial fibrillation). This leads to increased stress for the patient.

German Patent Application No. DE 103 09 245 A1 discloses a device for locating a lesion in a biological tissue portion, wherein the electrical excitation signals are applied to the tissue portion and electrical response signals are measured at a number of measurement locations over the surface of the tissue portion, said response signals being produced on account of the excitation signals there. A distribution of electric dipole moments is reconstructed on the basis of the response signals, and the spatial position of the distribution is output. A classification of the lesion as a benign or malignant lesion can be performed on the basis of these dipole moments and position thereof. By means of the known method, however, it is not possible to determine a local thickness of the tissue portion, since merely surface properties are detected.

U.S. Publication No. 2014/0324085 describes an ablation method in which energy for the ablation is introduced into the tissue by means of an ultrasound transducer. The ultrasound transducer is also used to determine the size of the lesion produced by the ablation. The known ablation method utilizes a complex and costly electronics set-up and transducer technology for the generation and evaluation of the ultrasound signal.

U.S. Publication No. 2015/0209551 likewise describes an ablation method by means of ultrasound. In the known method, the position of the catheter relative to the target site of the treatment additionally is determined by means of an imaging coil and a magnetically imaging system, with which the imaging coil can be detected. The known method is too imprecise to determine the depth of the lesion or the thickness of the tissue.

U.S. Publication No. 2015/196202 discloses a method for determining a lesion depth which is based on a measurement of the reduced mitochondrial nicotinamide adenine dinucleotide (NADH) fluorescence intensity of the illuminated heart tissue. The fluorescence intensity, however, can only be measured in a time-delayed manner. An optical querying method is also disclosed in International Application No. WO 2014/163974, which is limited to signal output in the single-digit millimeter range.

Applications of NMR sensors (NMR—nuclear magnetic resonance) are disclosed in documents such as U.S. Pat. No. 6,704,594, U.S. Publication No. 2005/0021019, U.S. Publication No. 2004/0158144 and U.S. Pat. No. 8,260,399.

The present invention is directed at overcoming one or more of the above-mentioned problems.

### SUMMARY

It is desirable to further increase the accuracy of the determination of a local tissue property, for example the lesion depth or the local tissue thickness. Further, a local treatment of the tissue, for example the ablation, should not be compromised by this determination. A further objective of the improvement is to provide a quickly and easily and economically determinable criterion, with which the progress of the treatment can be assessed.

An object of the present invention thus lies in creating a device with which a local tissue property, for example a lesion depth or tissue thickness, can be determined precisely, quickly, easily and economically and which has low interaction with the local tissue treatment. An additional object lies in describing a corresponding simple method for this purpose.

At least the above object is achieved by a device for determining (detecting) a local property of a biological tissue, said device comprising:
a flexible, elongate, preferably hollow cylindrical shaft,
a data processing device, and
a nuclear magnetic resonance (NMR) sensor, which is arranged at the distal end of the shaft and is connected to the data processing device, wherein the NMR sensor comprises a first sensor element for generating a static magnetic field and a second sensor element for generating a magnetic alternating field,
   wherein the distal end of the shaft can be arranged adjacently to the point of the tissue to be measured, wherein the data processing device is designed to determine a local tissue property at this point on the basis of a signal of the NMR sensor transmitted to the data processing device. On the basis of the local tissue property (for example local tissue cross-section and/or type of tissue and/or proportion of muscle tissue and/or composition of the tissue) determined by means of the device according to the invention, the progress of an ablation can be determined by the data processing device, for example by ascertaining the local depth of the lesion. The NMR sensor is preferably connected non-releasably to the distal end of the shaft.

The device may be an ablation catheter.

The data processing device can also be designed to determine the progress of formation of a lesion.

The inventors have identified that the properties of the tissue and change thereof as a result of an ablation, in particular in respect of temperature, tissue type (muscle tissue, fatty tissue), composition (for example water content) and/or the dimensions, can be detected by means of nuclear magnetic resonance (NMR). In particular, the amplitude of the measured nuclear magnetic resonance signal can be used to determine the size of the lesion area, i.e., the dimensions thereof.

In accordance with the present invention, the temperature of the adjacent point of the tissue, the thickness of the adjacent point of the tissue (in particular in the direction of the longitudinal axis of the shaft), the lesion depth (i.e., the depth of the lesion at the adjacent point of the tissue in the direction of the longitudinal axis of the shaft), the lesion size (i.e., the dimensions of the lesion at the adjacent point of the tissue in a direction perpendicular to the longitudinal axis of the shaft), information relating to contact between the ablation catheter and tissue (for example the compression of the tissue or the contact force based on the density of the tissue), the amount of tissue surrounding the distal end of the shaft, and the composition of the adjacent point of the tissue, in particular the water content thereof, the proportion of muscle tissue at the adjacent point of the tissue, the fat content thereof and/or proton density thereof, can be determined as local tissue property, for example. Here, the determination of a number of the above-mentioned tissue properties is also possible. Furthermore, the determination of a local tissue property in accordance with the invention also includes the determination of the change in the particular tissue property during the course of the (ablation) treatment or the measurement. The point of the tissue adjacent to the device according to the invention comprises a surface of the tissue at the point and a volume region of this tissue adjoining this surface in which an NMR excitation by the NMR sensor is performed, as described below in greater detail.

In order to generate the fundamentally known NMR signal, at least two components are required, specifically a static magnetic field, with the spins of protons for example being oriented in accordance with the field lines of said static magnetic field, and a magnetic alternating field, by which the spins are excited from their state of equilibrium. The static magnetic field is generated by the first sensor element, whereas the magnetic alternating field is produced by the second sensor element. In accordance with the present invention, the first and the second sensor element are arranged at the distal end of the flexible shaft, which for example is introduced into the body of a human or animal via the blood vessels and can be arranged in the immediate vicinity of the tissue point to be measured or the tissue region to be measured, where for example the ablation is performed. Here, preconditions for the excitation are field components of the magnetic alternating field oriented perpendicularly to the field lines of the static field. The frequency at which the spins are deflected is dependent on the magnitude of the magnetic flux of the static magnetic field. The following relationship applies for the resonance condition

$${f}_{L} = \frac{γ}{2 π} B$$

with γ as the gyromagnetic ratio (for ¹H protons: γ=267,513*10⁶ l/sT) and the magnetic flux density B.

Following the excitation, the relaxation time of the excited nuclear spins or the course over time of the oscillation amplitudes of the exciting magnetic alternating field can be measured. Tissue boundaries are noticeable during the measurement by a sudden change in the aforesaid measurands. Whereas blood, for example, has a long relaxation time with its high water content, tissue components with a lower water content have a comparatively short relaxation time. These different relaxation times are decisively responsible for the high soft tissue contrast of the NMR signal and, in the event of a local coding of the signals by means of magnetic field gradients, enable a local assignment of the tissue types and therefore of the thickness of the tissue.

It is advantageous if the NMR excitation by the NMR sensor occurs substantially in a conical volume about an axis in the spatial direction starting from the distal end of the flexible shaft. The conical volume is given from the course of the magnetic field lines of the static magnetic field in relation to the field lines of the magnetic alternating field. With suitable arrangement of the sensor elements, the two magnetic field components are arranged primarily perpendicularly to one another in a conical cylinder. In the volume outside the cylinder, the magnetic field lines run parallel to one another to the greatest extent and therefore do not contribute to the NMR signal. The excitation cone preferably has an opening angle (angle between two opposite lateral lines of the cone) of at most 180°, preferably at most 90°. It is particularly preferred if the axis extends in the distal direction from the distal end of the flexible shaft. It is furthermore advantageous if the excitation cone of the NMR excitation can be oriented in respect of the surface of the tissue point to be measured such that the axis of the excitation cone extends perpendicularly to the tissue surface at the point to be measured.

The penetration depth for the NMR signal in the tissue to be examined (i.e. the height of the excitation cone) is dependent on the frequency of the alternating field or bandwidth thereof. For ¹H protons, frequencies in the megahertz range are provided as resonance frequency in the direct vicinity of the magnet (distance <3 mm), and are reduced to 1 kHz up to a distance of 35 mm. If, for example, a spherical magnet is used as static magnet (first sensor element) with 1 T maximum magnetic flux density at the surface, spins at a distance of up to 3 mm are excited by high frequencies in the megahertz range. With frequencies of 1 MHz to 1 kHz, spins are excited at a distance of up to 34 mm. With use of a lower magnetic flux density, the penetration depth decreases in this frequency range in accordance with the resonance condition.

With a broadband excitation pulse of this kind of the frequency range corresponding to the desired excitation depth, a volume excitation of the spins is achieved in the distal direction starting from the distal end of the shaft. Depending on their distance, the excited spins send a response signal with the corresponding resonance frequency, such that a one-dimensional spatial resolution by means of a Fourier analysis is possible as a function of the distance.

At least one material from the group comprising neodymium, hardened steel, ferrites, aluminum-nickel-cobalt alloys, bismuth-manganese-iron alloys (bismanol) or samarium-cobalt alloys is preferably used as material for the first sensor element for generation of a static magnetic field. The magnetic flux densities at the surface of the first sensor element lie preferably in a range of 0.5 T to 1.5 T (inclusive).

In a preferred exemplary embodiment, the first sensor element is formed as a permanent magnet, which for example is spherical or cuboid-shaped, or as a coil. With useful example of a spherical solid-state permanent magnet (neodymium, with for example 1 T flux density at the surface), the flux density decreases with the distance from the catheter approximately with the third power and assimilates that of a rod magnet. The spherical or cuboid-shaped design of the solid-state magnet allows a simple orientation of the static magnetic field. In addition, a directed excitation cone can be produced. In a preferred exemplary embodiment, the material of the permanent magnet is not electrically conductive, so as to avoid eddy currents, which are induced by the magnetic alternating field. Permanent magnets for example made of neodymium and most other materials for example have the permeability of air. The permeability, however, can also be 2, 4 or up to 8 in the case of aluminum-nickel-cobalt, whereby the efficiency of the second sensor element for generation of the magnetic alternating field is increased accordingly. In order to generate a permanent magnetic field which has weaker non-linear behavior compared to a spherical magnet, the permanent magnet can also be provided in the form of a horseshoe magnet, wherein preferably the arms of the horseshoe magnet run parallel to the longitudinal axis of the shaft.

It is also advantageous if the second sensor element is formed as a coil. In order to generate and receive the magnetic alternating field, circular conductor coils are preferably used. In the advantageous frequency band of MHz-kHz, a winding number of at most 10 is preferred, with a winding number of 5 to 10 being particularly preferred. In the exemplary embodiment in which the first sensor element is formed as a permanent magnet, the coil is preferably wound around the first sensor element. In order to keep the opening angle of the excitation cone for the signal generation as small as possible in the case of a horseshoe permanent magnet as first sensor element, the coil for the magnetic alternating field can be arranged between the two arms of the horseshoe magnet. The additional use of a ferromagnetic, non-electrically conductive coil core in order to increase the field strength both the first and of the second sensor element is also advantageous. The field lines of the permanent magnet run preferably perpendicularly to the axis of the shaft at the distal end thereof, and the field lines of the magnetic alternating field run preferably along the axis of the shaft.

The outer dimensions (length optionally in the direction of the longitudinal axis, width or diameter optionally transverse to the longitudinal axis, optionally depth) of the first and second sensor elements are between 0.5 mm and 5 mm, preferably between 1 mm and 3 mm—defined by the available space in/at the distal end of the shaft.

Since the device according to the present invention in one exemplary embodiment can be used for ablation, it is advantageous if an electrically conductive surface in the form of a metallized shaft tip is arranged at the distal end of the shaft. By means of the shaft tip, electrical current is introduced into the adjacent tissue, which generates a tissue lesion. In its function as ablation surface, this metal surface shields against electromagnetic waves, in particular the magnetic component thereof. In order to make the metal surface permeable for magnetic fields and therefore for the generation of NMR signal and in particular for the receipt of NMR signals, at least one continuous slot-shaped recess is provided in the metal shaft tip, for example in the form of a cross slot, in order to avoid the formation of eddy currents in the shaft tip. In an alternative exemplary embodiment the shaft tip is embodied as a helix antenna in order to reduce the described shielding effect. Depending on the orientation of the magnetic field lines of the static magnetic field, the slot-shaped recess of the helix antenna must be formed in such a way that the magnetic field lines of the alternating field in the desired excitation area run perpendicularly to the field lines of the static magnetic field of the first sensor element.

A simple exemplary embodiment for an NMR sensor is provided on account of the geometric constraints of the shaft when, as second sensor element, a coil for generation of the magnetic alternating field is wound around for example a spherical permanent magnet as first sensor element, which generates the static magnetic field.

In one exemplary embodiment of the present invention, the NMR sensor is pivotable and/or rotatable relative to the shaft by means of a corresponding control mechanism by means of at least one pull cable fastened to the NMR sensor, so as to orientate the axis of the excitation cone of the NMR sensor in a direction perpendicular to the surface of the tissue with the tissue point to be examined. The at least one pull cable is preferably fastened to the outer periphery of the first sensor element. In particular, the geometry of the NMR sensor with a spherical permanent magnet as first sensor element allows the rotation of the combination of permanent magnet and electromagnet in the direction that is of particular interest for the signal output, for example when the shaft is arranged at its distal end at a relatively flat angle in relation to the tissue surface. In exemplary embodiments, two pull cables arranged opposite one another (i.e. distance from one another at an angle of 180°) or pull cables distanced in each case at an angle of 90° can be provided, which pull cables preferably pass through the shaft and can be actuated from outside, so as to pivot and/or rotate the NMR sensor in relation to the longitudinal axis of the shaft. In addition, the shaft can be rotated about its longitudinal axis.

In a further exemplary embodiment, the NMR sensor can be supported on a substrate which has a first portion with a high or higher elasticity, preferably in the direction of the longitudinal axis of the shaft, and a second portion with a lower elasticity as compared to the first portion. The first portion and a second portion are preferably arranged side by side in a direction transverse to the longitudinal axis of the shaft. If the NMR sensor is pivoted in relation to the longitudinal axis of the shaft, the first portion brings about a restoring force. The orientation of the NMR sensor is hereby facilitated, and the device is made simpler, since only a single pull cable is necessary. The second portion of the substrate with the lower elasticity (or higher rigidity) can consist for example of a plastic, such as TPU (thermoplastic polyurethane), PEEK (polyether ether ketone), polyether block amide (PEBA, such as Pebax), or LCP (liquid crystal polymer). The first portion of the substrate with the high or higher elasticity can consist for example of a foamed plastic or silicone or can have a leaf spring-like structure, which for example is manufactured from plastic.

On account of the comparatively strong and non-linear static magnetic field gradient of a spherical solid-state magnet, the excited spins will de-phase within a short period of time, and detection of the signal response will be hindered accordingly. This circumstance can be counteracted by the excitation by means of magnetic alternating field pulses by the NMR sensor and by use of a spin echo, for example in that a further pulse is sent after a 90° excitation pulse, which further pulse rotate the spins through 180°, i.e. reverses them. The duration of an alternating field pulse is between 1 and 50 milliseconds, preferably between 1 and 20 milliseconds.

At least the above object is achieved with similar advantages also by a catheter, in particular an ablation catheter, comprising a device as described above. Besides the determination of the tissue property, further components arranged in or on the catheter or components connected to the catheter can facilitate the positioning at a suitable therapy site. Components of this kind can, for example, be a device for navigation, wherein the catheter in this case is connected for example to a magnetometer or an electric field meter. The field for position determination generated extracorporeally by the magnetometer or the electric field meter is designed here in such a way that it does not influence the NMR signal. Further components at the catheter for positioning at a suitable therapy site are electrodes arranged on the catheter in the form of ring electrodes or mini electrodes, which make it possible to detect local electrical signals. Local cell activities in the context of lesion formation and the impulse conduction system can thus be assessed. A force sensor or a plurality of force sensors can be arranged on the catheter (for example at the distal end of the shaft) as a further component for monitoring lesion development, with the transducer of said sensor(s) being based usually on electromagnetic or fiber-optic principles. The electromagnetic interaction of the one or more corresponding components with the NMR sensor must be taken into consideration. For example, the frequencies of the electromagnetic fields can be coordinated, the interference fields can be switched off during the measurement, or corresponding filters or signal processing elements can be used. With integration of the second sensor element in an ablation electrode arranged at the distal end of the shaft, the second sensor element can also be used to emit energy during the ablation, whereby the energy output is optimized.

At least the above object is also achieved by a method for determining a local property of a biological tissue, in which method, following excitation by an NMR sensor arranged at the distal end of a flexible shaft, adjacently to the point of the tissue to be measured, an NMR response signal (referred to hereinafter as NMR signal for short) of the tissue is generated and the local tissue property is determined on the basis of this NMR signal. The evaluation of the NMR signal corresponds in principle to the evaluation of imaging MRT signals. The received NMR signals are characterized in the data processing device both via their amplitude and their phase. Via the phase, it is possible to quantify the temperature change over time. The amplitude is determined by the proton density of the tissue and the transverse (T2) and longitudinal (T1) relaxation times characteristic for tissue types. The T1 time is additionally depending on the temperature of the tissue. An increase in the temperature simultaneously increases the T1 relaxation time of the area in question, which leads directly to a reduction of the NMR signal. The occurrence of a lesion by the introduction of thermal energy in the medium-term changes the water content of the tissue, which leads to a change in the density of the free protons and a change in the T2 relaxation time.

The method according to the present invention has the advantages explained above in relation to the device. The excitation by means of NMR sensor and the determination of the local tissue property on the basis of the transmitted NMR signals are controlled by means of the data processing device.

With regard to the local properties of the biological tissue determinable with the method according to the invention, reference is made to the above explanations provided in relation to the device according to the present invention.

As already described above, the axis of an excitation cone of the NMR sensor is oriented substantially perpendicularly to the tissue surface prior to the generation of the NMR signal in one exemplary embodiment of the method according to the invention. The orientation is particularly preferably performed:

by actuating at least one pull cable fastened to the NMR sensor, for example by means of a control mechanism arranged on the shaft, such that a pivoting and/or rotation of the NMR sensor relative to the longitudinal axis of the shaft is brought about, and/or

by rotating the shaft. Additionally or alternatively, the distal end of the shaft can be displaced in the direction of the longitudinal axis of the shaft in such a way that the distal end of the shaft bears against the surface of the tissue to be measured.

In a further exemplary embodiment, intermittently between the determination of the local tissue property on the basis of the NMR signal, a shaft tip arranged at the distal end of the shaft is supplied with a current or a voltage is applied to the metal shaft tip, such that the tissue is ablated by means of the shaft tip and a lesion is created in the tissue.

In a further exemplary embodiment of the method according to the present invention, as explained above, the excitation is achieved by means of magnetic alternating field pulse by the NMR sensor and by use of a spin echo method, in which for example a further pulse is sent after an excitation pulse (also referred to as a 90° excitation pulse), with said further pulse rotating the spins through 180°.

At least the above object is also achieved by a computer program product for determining a local property of a biological tissue, said computer program product comprising program code means for executing a computer program following implementation thereof in a data processing device. The program code means are intended to execute the above-described method following the implementation in the data processing device. The computer program product according to the present invention has the advantages explained above in relation to the method according to the invention.

Further features, aspects, objects, advantages, and possible applications of the present invention will become apparent from a study of the exemplary embodiments and examples described below, in combination with the Figures, and the appended claims.

### DESCRIPTION OF THE DRAWINGS

The present invention will be explained hereinafter on the basis of exemplary embodiments and with reference to the drawings. Here, all features described and/or shown in the drawings form the subject matter of the present invention, individually or in any combination, and also independently of their summary in the claims and the dependency references of the claims.

The drawings show schematically:

FIG. 1 shows a catheter according to the present invention in a view from the side,

FIG. 2 shows a device according to the present invention in a view from the side,

FIG. 3 shows a first exemplary embodiment for the primary realization of the NMR sensor of the device according to FIG. 2,

FIG. 4 shows a second exemplary embodiment for the primary realization of the NMR sensor of the device according to FIG. 2,

FIG. 5 shows a third exemplary embodiment for the primary realization of the NMR sensor of the device according to FIG. 2,

FIG. 6 shows a second exemplary embodiment of a device according to the present invention in a view from the side including the magnetic field lines of the first sensor element,

FIG. 7 shows the NMR sensor of the device according to FIG. 6 in a view from the side,

FIG. 8 shows the shaft tip of the device according to FIG. 6 including the magnetic field lines of the second sensor element in a view from the side,

FIG. 9 shows a second exemplary embodiment of a shaft tip of the device according to FIG. 6 in a view from above,

FIG. 10 shows a third exemplary embodiment of a shaft tip of the device according to FIG. 6 in a view from the side,

FIG. 11 shows the shaft tip according to FIG. 10 in a view from above,

FIG. 12 shows a third exemplary embodiment of a device according to the present invention in a view from the side including the magnetic field lines of the first sensor element,

FIG. 13 shows the NMR sensor of the device according to FIG. 12 in a view from the side,

FIG. 14 shows the shaft tip of the device according to FIG. 12 including the magnetic field lines of the second sensor element in a view from the side,

FIG. 15 shows a second exemplary embodiment of a shaft tip of the device according to FIG. 12 in a view from the side,

FIG. 16 shows the shaft tip according to FIG. 10 in a view from above,

FIGS. 17-22 show the orientation of the excitation cone by means of rotation of the NMR sensor of the device according to FIG. 9,

FIG. 23 shows a further exemplary embodiment of an NMR sensor of a device according to the present invention in a view from the side,

FIG. 24 shows the magnetic field lines of the second sensor element of the NMR sensor according to FIG. 23,

FIG. 25 shows the magnetic field lines of the first sensor element of the NMR sensor according to FIG. 23,

FIGS. 26-27 show the orientation of the excitation cone by means of rotation of the NMR sensor according to FIG. 23, and

FIG. 28 shows the excitation of the protons by means of the NMR sensor in accordance with the sin echo method in the time domain and the frequency domain.

### DETAILED DESCRIPTION

The design and the operating principle of a catheter according to the present invention or of a device according to the present invention comprising a shaft will be explained hereinafter on the basis of an ablation catheter which is used for intracardiac ablation. The present invention, however, is not intended to be limited to this example. The design and the operating principle of a catheter according to the present invention all of a device according to the present invention can be transferred analogously to catheters/devices for other treatments or other tissues, wherein the determination of the local tissue property, for example the local thickness or local lesion depth, is of significance.

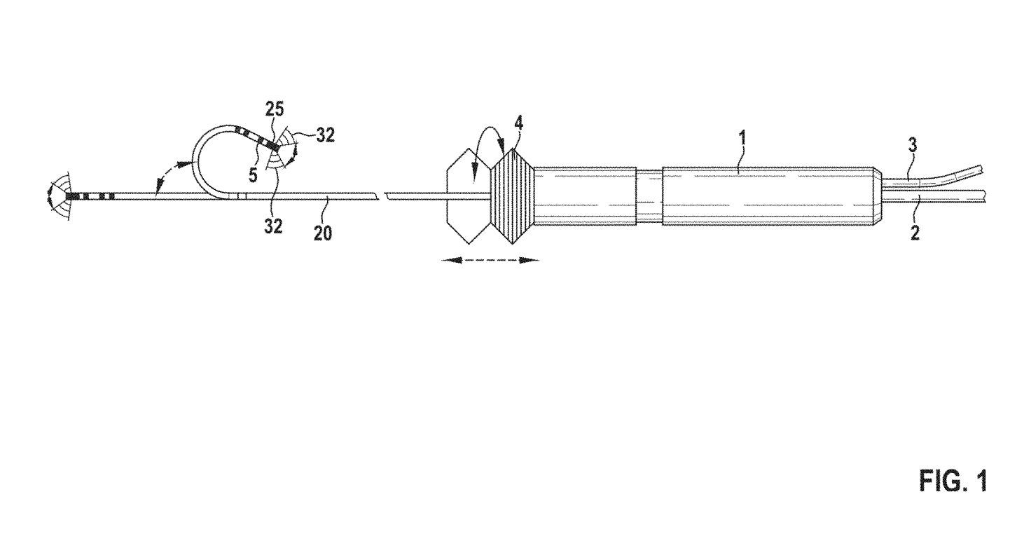

FIG. 1 shows an exemplary embodiment of a catheter according to the present invention with a handgrip 1, at least one electrical and/or optical signal line 2 for the transmission of signals from and/or to the at least one or sensor or sensor element, mounted on the catheter, and/or the at least one electrode, a flush line 3, a control mechanism 4, and an inner shaft 20. The inner shaft 20 as part of the device according to the present invention. For ablation, the inner shaft 20 is inserted into the body of the patient, for example along the blood vessels of the patient, until the distal end of the inner shaft 20 bears against the desired point of the heart muscle tissue which is to be ablated. In order to detect the electrical cardiac activity, at least one electrode 5 is provided at the distal end of the inner shaft 20. In the embodiment shown in FIG. 1, the electrode 5 is formed as a ring electrode. A mini electrode arranged within the distal tip of the inner shaft 20 is likewise conceivable. By means of the control mechanism 4, the distal end of the inner shaft 20 can be deflected for example via a push-pull mechanism, as is illustrated by means of the dashed arrows. Additionally, as will be described below in greater detail, the excitation cone 32, by means of a rotational movement of the control mechanism 4, can be oriented relative to the tissue to be examined and to be ablated. Alternatively to the manual control by means of the control mechanism 4, a bidirectional automated control can be applied.

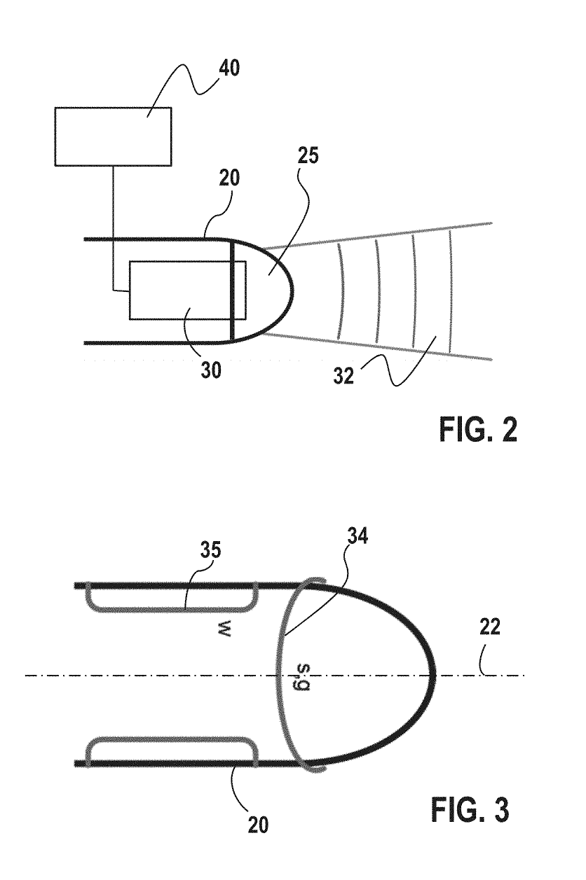

At the distal end of the inner shaft 20 (see FIG. 2), an electrically conductive shaft tip 25 is provided, which is connected to an electrical circuit. The connections are disposed on the inner side of the shaft tip 25 and are guided through the inner shaft 20. For the ablation, the shaft to 25 is exposed to an electrical high-frequency current via a signal line 2. As a result of the contact of the shaft tip 25, the high-frequency current also passes into the heart muscle tissue bearing against the shaft tip 25 and is hereby destroyed.

In order to assess the progress of the lesion formation or the ablation, the catheter according to the invention has an NMR sensor at the distal end of the inner shaft 20. This NMR sensor 30 is connected to a data processing device 40 (for example a (micro)processor or a computer) arranged outside the body of the patient. The assessment of the progress of the ablation is implemented by the NMR sensor 30 and is controlled by the data processing device 40. Before the treatment is started and at the end of each treatment step, the NMR sensor 30 is activated by the data processing device 40 and excites, in an excitation cone 32, the protons of the heart muscle tissue 50 disposed in the excitation cone 32. By superimposing a static magnetic field and a magnetic alternating field, the spins of the protons are oriented and brought out of their state of equilibrium. The NMR signal emitted by the protons as they return to the state of equilibrium is detected by the NMR sensor 30 and transmitted to the data processing device 40. This device, on the basis of the difference between amplitude and phase of the NMR signal before the onset of the ablation and the last-measured NMR signal, calculates in particular the difference in the amplitude, for example the reduction in the thickness of the heart muscle tissue at the point disposed in the excitation cone 32, and on this basis also calculates the lesion depth. As soon as a sufficient lesion depth is reached, the treatment at this point can be terminated and as applicable continued at another point. The limit value for the amplitude and/or phase change of the NMR signal at which the treatment is terminated can be defined experimentally.

The catheter according to the present invention thus enables a precise assessment of the progress of the lesion formation or the ablation in a simple way.

As has already been explained above, the NMR sensor 30 has a first sensor element 34, which generates a static magnetic field, and a second sensor element 35, which produces a magnetic alternating field. Here, the field lines of the static magnetic field of the first sensor element 34 and the field lines of the magnetic alternating field of the second sensor element 35 must be arranged perpendicularly to one another at least in the excitation cone 32. Three fundamental exemplary embodiments for the realization of the first and second sensor element are shown with reference to FIGS. 3 to 5.

In the exemplary embodiment according to FIG. 3, the first sensor element 34 is embodied as a coil, the magnetic field lines of which run parallel to the (longitudinal) axis 22 of the inner shaft 20. The second sensor element 35 is likewise embodied as a coil, wherein the magnetic field lines of this coil run perpendicularly to the axis 22. In an alternative exemplary embodiment, both the first sensor element 34 and the second sensor element 35 can each be embodied as a coil, wherein in this case the magnetic field lines of the first sensor element run perpendicular to the axis 22 of the inner shaft 20, and the magnetic field lines of the second sensor element run parallel to the axis 22 of the inner shaft 20.

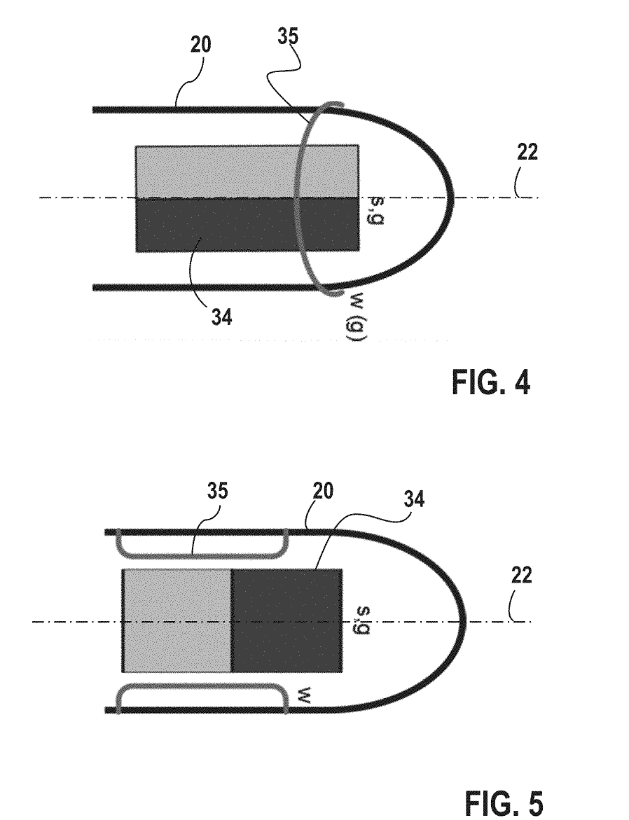

In the exemplary embodiments shown in FIGS. 4 and 5, the first sensor element 34 is embodied as a permanent magnet. By contrast, the second sensor element 35 is embodied as a coil. In the exemplary embodiment shown in FIG. 4, the magnetic field lines of the first sensor element 34 run perpendicularly to the axis 22 of the inner shaft 20, and in the exemplary embodiment shown in FIG. 5 parallel to the axis 22 of the inner shaft 20. Accordingly, the magnetic field lines of the second sensor element 35 in the exemplary embodiment shown in FIG. 4 run parallel, and in the exemplary embodiment shown in FIG. 5 run perpendicular to the axis 22 of the inner shaft 20.

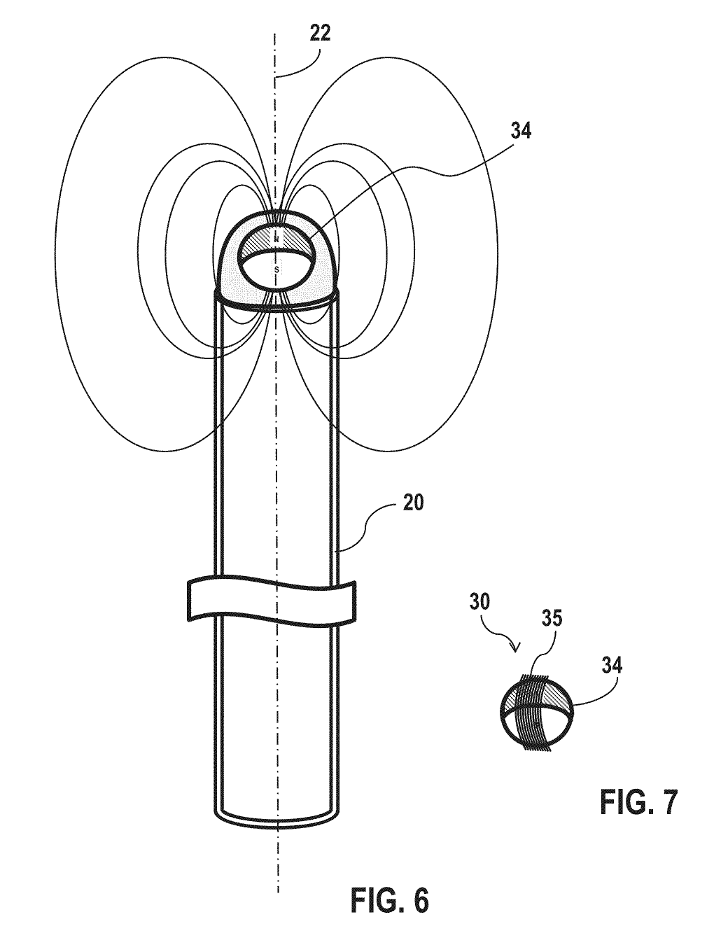

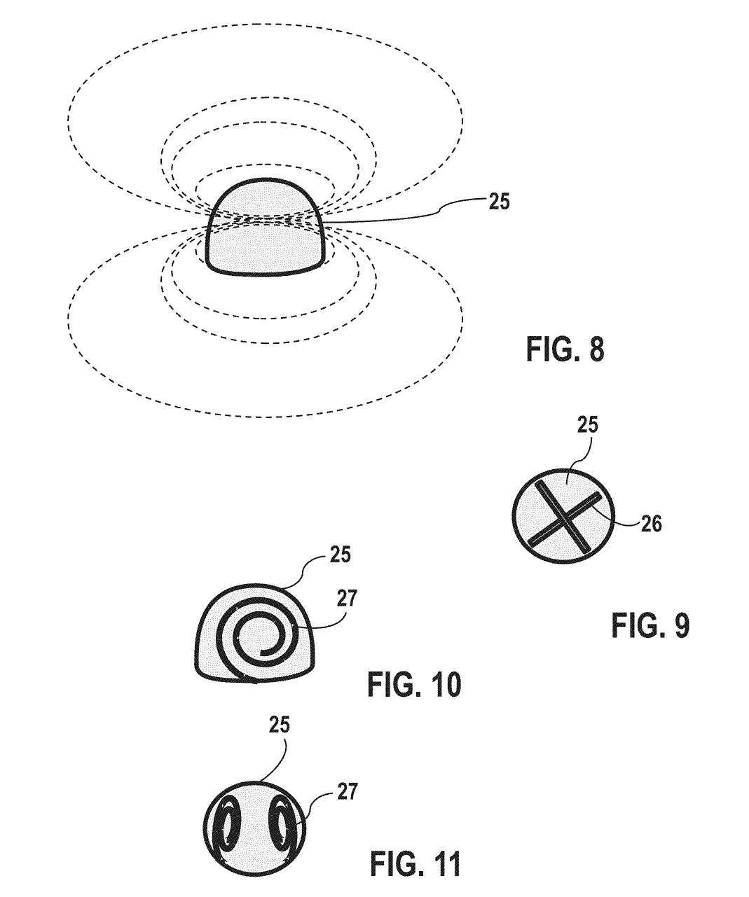

The exemplary embodiment shown in FIGS. 6 and 7 corresponds to the principle shown in FIG. 5, wherein the first sensor element 34 is spherical. The second sensor element 35 is a coil which is wound around the spherical first sensor element and which for example is made from neodymium. The arrangement formed of first sensor element 34 and second sensor element 35 is shown in FIG. 7. The first sensor element for example has a diameter of 2 mm. The magnetic flux density of the first sensor element is for example 1 T at the surface. The magnetic field lines of the first sensor element 34 are shown in FIG. 6, whereas the magnetic field lines of the second sensor element are shown in FIG. 8 (see dashed lines).

In order to avoid the formation of shielding circuit currents in the metal shaft tip 25, said shaft tip has a cross slot 26, which passes through the shaft tip 25. The slot of the cross slot for example has a width of 0.1 mm (see FIG. 9). Alternatively, a continuous spiraled slot 27 is provided laterally on the shaft tip 25. The axis of the spiral, as can be inferred from FIGS. 10 and 11, runs at an angle of at least 70° to the axis 22 of the inner shaft 20. The spiraled slot 27 likewise has a width of 0.1 mm, for example.

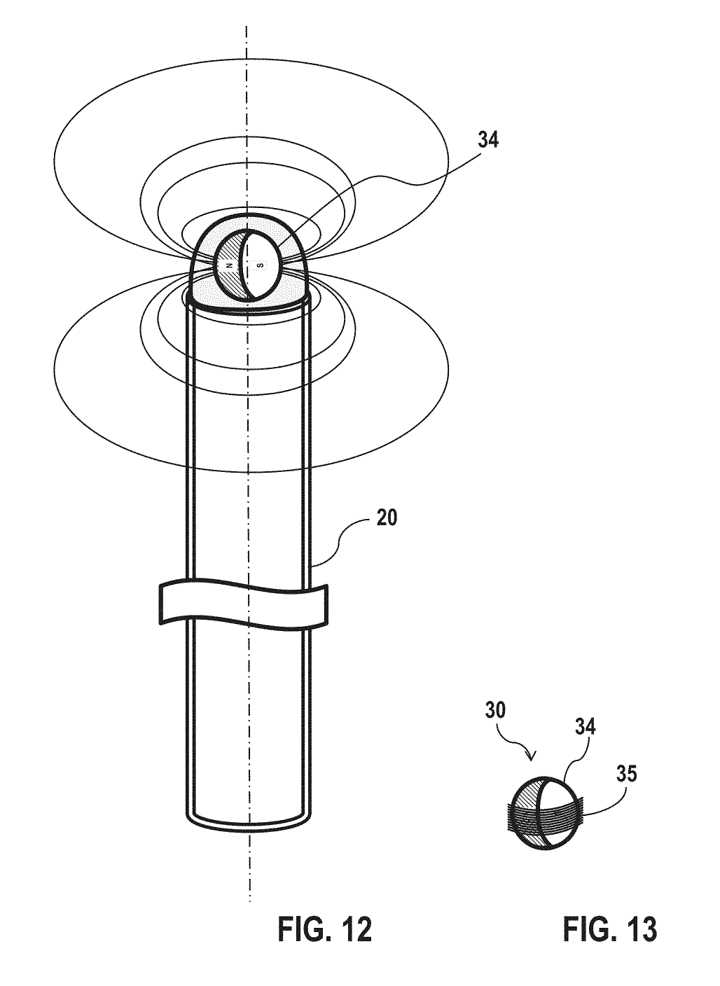

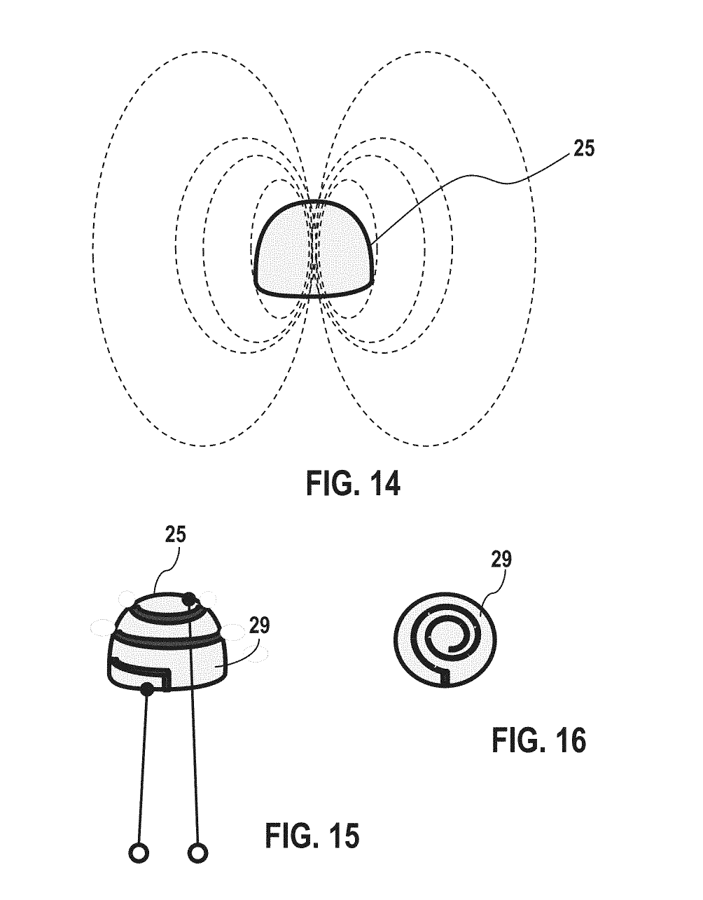

The exemplary embodiment shown in FIGS. 12 and 13 corresponds to the principle shown in FIG. 4 of the arrangement of the first and second sensor element, wherein in this exemplary embodiment as well the first sensor element 34 is formed as a spherical neodymium permanent magnet. The second sensor element 35 is a coil which is wound around the spherical first sensor element 34. The arrangement formed of first sensor element 34 and second sensor element 35 is shown in FIG. 13. The first sensor element for example has a diameter of 2 mm. The magnetic flux density of the first sensor element 34 is for example 1 T at the surface. The magnetic field lines of the first sensor element 34 are shown in FIG. 12, whereas the magnetic field lines of the second sensor element 35 are shown in FIG. 14 (see dashed lines).

In order to avoid the formation of shielding circuit currents in the metal shaft tip 25 in the exemplary embodiment shown in FIG. 12, said shaft tip, as shown in FIGS. 15 and 16, is embodied as a helix antenna 29. The number of helix turns is limited by the length of the metal catheter tip and lies preferably in the range of from 5 to 10 turns. In the region of the tapering catheter tip, the turns of the helix antenna 29 can be formed in an equiangular or equidistant manner (Archimedes spiral) in order to increase the bandwidth of the antenna. The thickness of the wire or helix antenna is for example between 0.05 mm and 0.5 mm.

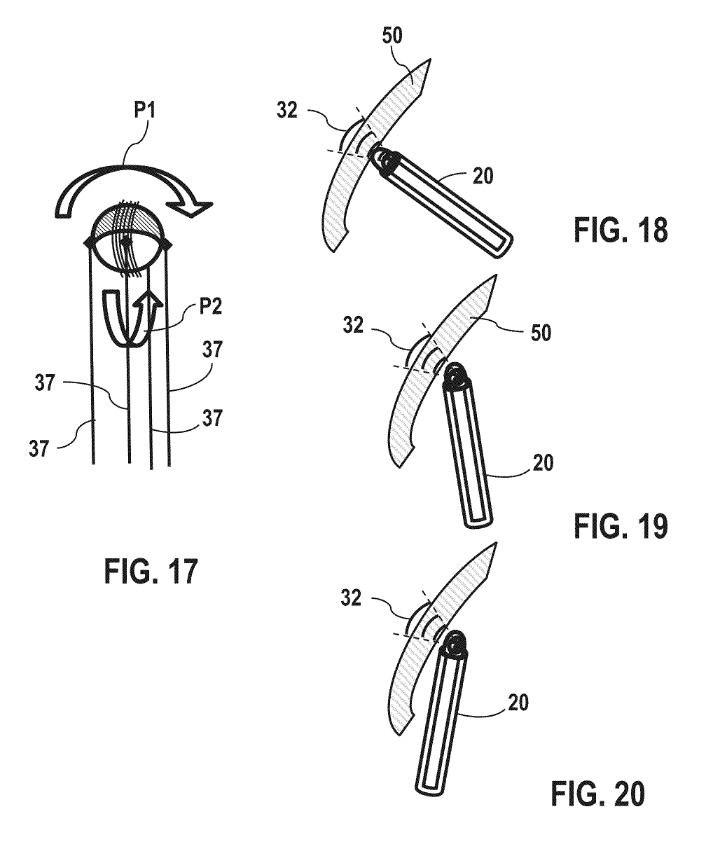

In order to orientate the NMR sensor 30 of the exemplary embodiment shown in FIG. 6 such that the axis of the excitation cone 32 runs approximately perpendicularly to the surface of the heart muscle tissue at the point to be examined, four pull cables 37 are fastened to the periphery of the first sensor element 34. This is shown in FIG. 17. The four pull cables 37 are arranged at the periphery of the first sensor element 34 in such a way that they each enclose an angle of 90° with the adjacent pull cable 37. By pulling suitably on one or more pull cables 37, the movably mounted NMR sensor 30 can be rotated and/or pivoted (see arrows P1 and P2) about the center point or another point, preferably lying on the axis 22 of the inner shaft 20, within the first sensor element 34 and therefore in relation to the axis 22. The NMR sensor 30 can be mounted, for example, by means of a spherical shell element (not shown), wherein the NMR sensor is arranged in the spherical shell segment. Examples of an orientation of this kind in relation to the heart muscle tissue 50 are shown in FIGS. 18 to 20. In the variant of FIG. 18 the excitation cone 32 runs substantially parallel to the axis 22 of the inner shaft 20. In the constellation of FIG. 19, the axis of the excitation cone 32 runs for example at an angle of 30° to the axis of the excitation cone 32. FIG. 20 shows that, as a result of this manipulation, the excitation cone 32 can be pivoted relative to the axis of the inner shaft 20 such that the axis of the excitation cone encloses an angle of approximately 70° with the axis 22 of the inner shaft.

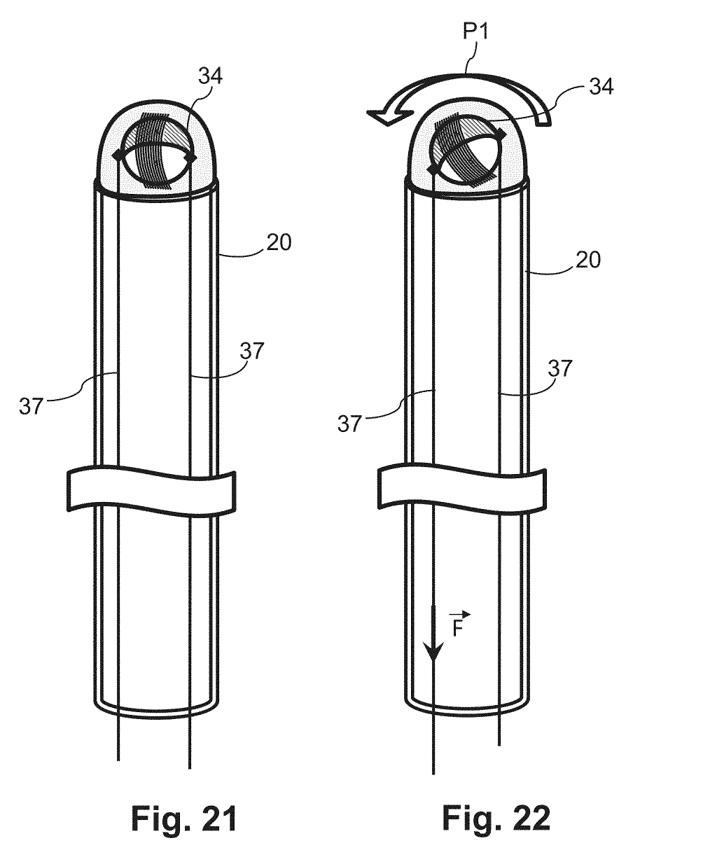

A similar manipulation can also be achieved by means of an arrangement in which only two pull cables 37 are provided, which are fastened to the periphery of the first sensor element 34, more specifically in a mutually opposed arrangement. An exemplary embodiment of this kind is shown in FIGS. 21 and 22. The arrow F arranged at one pull cable 37 represents the force (value and direction) which is applied by pulling on the pull cable 37 in order to rotate or pivot the NMR sensor 30 (see arrow P1) relative to the axis 22. In order to achieve the orientation of the excitation clone 32 in any (three-dimensional) direction, the inner shaft 20 can be rotated additionally about its axis 22.

The movement of the excitation cone is brought about preferably by means of the control mechanism 4.

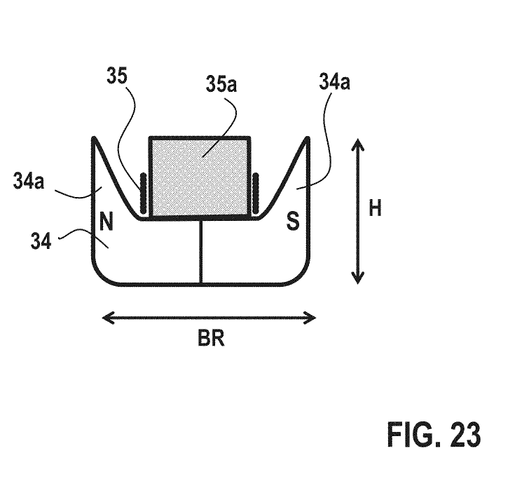

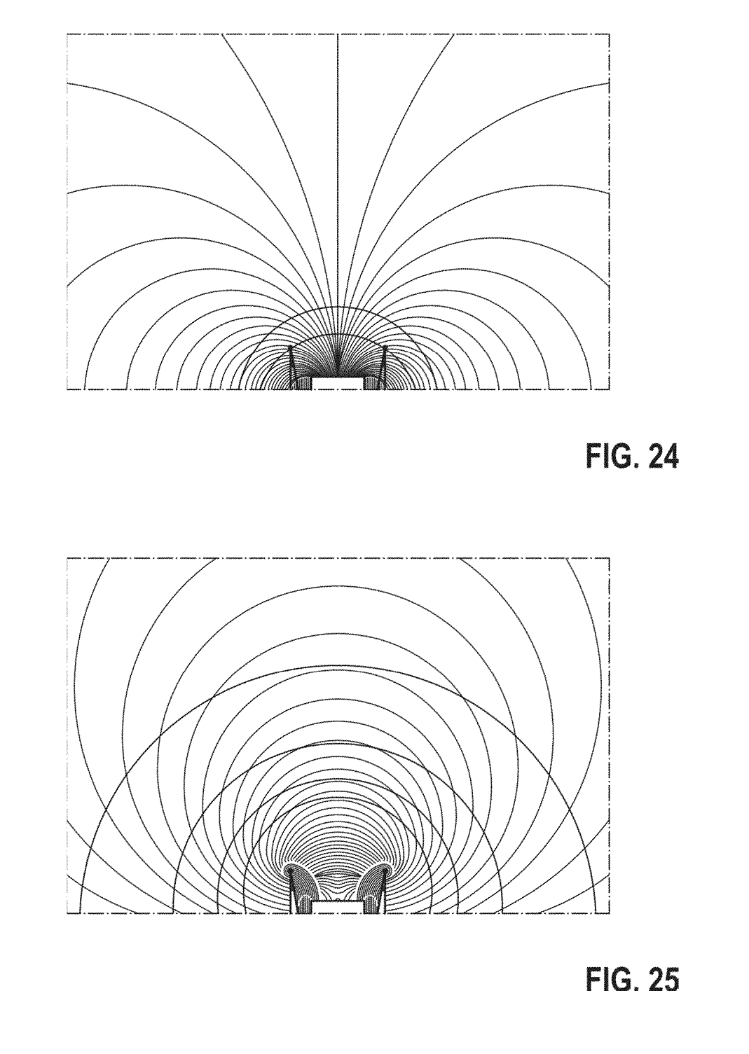

FIG. 23 shows a further exemplary embodiment of an NMR sensor 30, which has weaker non-linear behavior as compared to the above-described exemplary embodiments with the spherical permanent magnet. The first sensor element 34 is formed by a horse shoe-shaped permanent magnet, which is preferably made of neodymium. The first sensor element 34 for example has a width B of the base of 2 mm and a height H of the arms 34a of 1 mm to 2 mm. The magnetic field lines of the first sensor element are shown in FIG. 25 and run perpendicularly to the axis 22 of the inner shaft 20. In order to keep the opening angle of the excitation cone 32 as small as possible, the second sensor element 35 is embodied as a coil which is arranged between the arms 34a of the horseshoe-shaped first sensor element 34. In a preferred exemplary embodiment the second sensor element 35 has a ferromagnetic, non-electrically conductive coil core 35a, which increases the attained field strength. The magnetic field lines of the second sensor element 35 are shown in FIG. 24 and run parallel to the axis 22 of the inner shaft 20.

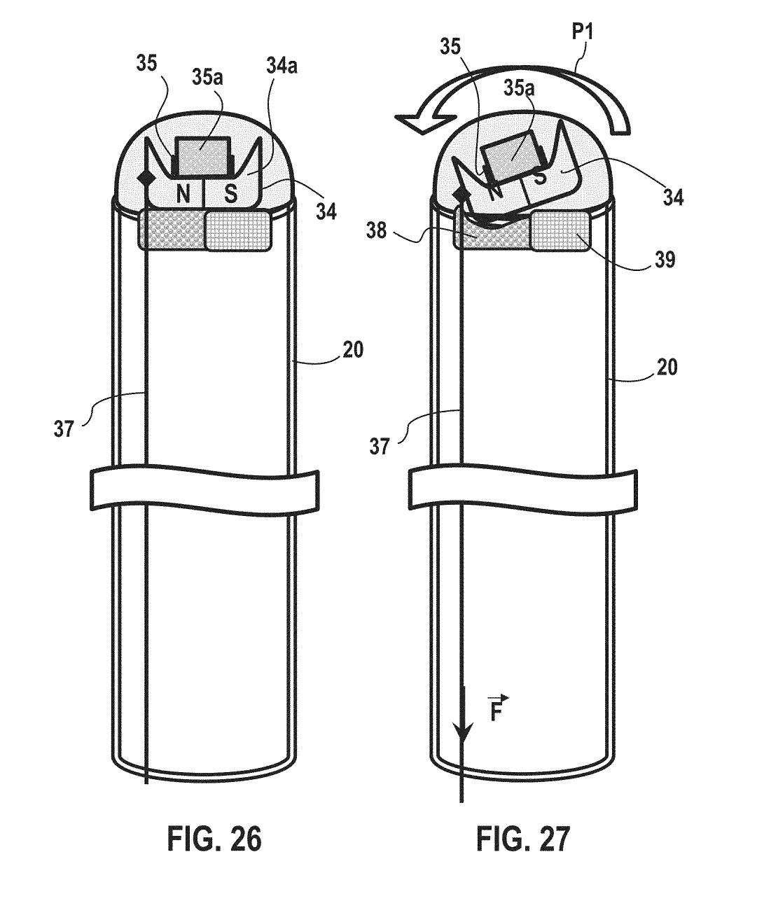

As is shown in FIGS. 26 and 27, the NMR sensor 30 is mounted on a substrate that is resilient at least in regions. The substrate comprises a first portion 38, which has a higher elasticity, and a second portion 39, which has a lower elasticity, wherein the first portion 38 and the second portion 39 are arranged side by side transversely to the longitudinal axis of the inner shaft 20. A pull cable 37 is also fastened to the outer side of an arm 34a of the first sensor element 34. By pulling on the pull cable (see the direction of the force F indicated by an arrow in FIG. 27), for example by means of the control mechanism 4, the NMR sensor is pivoted about an axis arranged perpendicular to the image of FIG. 27 (see arrow P1) and therefore also relative to the longitudinal axis of the shaft 20, such that the excitation cone can be oriented in relation to a tissue surface. As applicable, the inner shaft 20 is additionally rotated about its axis 22, in order to provide the orientation in any spatial direction. The resilient first portion 38 of the substrate causes a restoring force and causes the NMR sensor 30 to pivot back into the starting position shown in FIG. 26 when the tensile force F on the pull cable 37 is reduced.

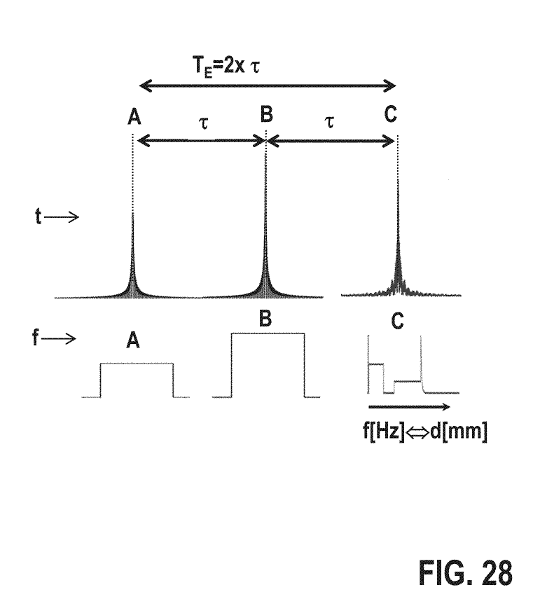

On account of the relatively strong and non-linear static magnetic field gradient of a first sensor element 34 formed as a spherical solid-state magnet, the excited spins will de-phase within a short period of time. This circumstance can be counteracted by means of spin echo methods, in which for example a further pulse is sent after a 90° excitation pulse, which further pulse returns the spins of the protons through 180° (see FIG. 28). Each magnetic field pulse is a broadband pulse over a frequency range of for example 1 kHz to 20 MHz The excitation with the pulses A and B as well as the NMR signal C from the tissue are shown in FIG. 28 at the top in the time domain and at the bottom in the frequency domain.

The present invention uses the known NMR technology in order to determine, in a simple and economical manner, the progress of a treatment or the size of a lesion, in particular the depth thereof in the tissue. With the solution according to the present invention, by means of the design of the NMR sensor 30, the NMR excitation can be limited to an excitation cone 32 having a small opening angle. The depth of the observation field can be influenced via the magnetic field parameters.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 handgrip of the catheter
2 signal line
3 flush line
4 control mechanism
5 electrode
20 inner shaft
22 axis (longitudinal axis) of the inner shaft
25 shaft tip
26 cross slot
27 spiralled sot
29 helix antenna
30 NMR sensor
32 excitation cone
34 first sensor element
34a arm of the horseshoe magnet
35 second sensor element
35a coil core
37 pull cable
38 first portion of the substrate
39 second portion of the substrate
40 data processing device
50 heart muscle tissue
A,B excitation pulse
BR width
C NMR signal
F force
H height
P1 arrow 1
P2 arrow 2
f display in frequency domain
t display in time domain

I/We claim:

1. An ablation catheter for determining a local property of a biological tissue, comprising:
a flexible shaft,
a data processing device, and
an NMR sensor, which is arranged at the distal end of the shaft and is connected to the data processing device, wherein the NMR sensor comprises a first sensor element for generating a static magnetic field and a second sensor element for generating a magnetic alternating field, wherein the distal end of the shaft can be arranged adjacently to the point of the tissue to be measured, wherein the data processing device is designed to determine a local property of the tissue at this point on the basis of a signal of the NMR sensor transmitted to the data processing device, and wherein the data processing device is also designed to determine the progress of formation of a lesion.

2. The ablation catheter according to claim 1, wherein the first sensor element is formed as a permanent magnet or as a coil.

3. The ablation catheter according to claim 2, wherein the permanent magnet is spherical or cuboid-shaped.

4. The ablation catheter according to claim 1, wherein the second sensor element is formed as a coil.

5. The ablation catheter according to claim 1, wherein a shaft tip arranged at the distal end of the shaft has at least one recess in the form of a slot or is embodied as a helix antenna.

6. The ablation catheter according to claim 1, wherein the NMR sensor is pivotable and/or rotatable relative to the shaft by means of at least one pull cable fastened to the NMR sensor.

7. The ablation catheter according to claim 1, wherein der NMR-Sensor is mounted on a substrate which has a first portion with a higher elasticity and a second portion with a lower elasticity as compared to the first portion, wherein the first portion brings about a restoring force when the NMR sensor is pivoted relative to the shaft.

8. The ablation catheter according to claim 1, wherein the NMR sensor is designed for excitation by means of magnetic alternating field pulses, wherein a further pulse is sent after a 90° excitation pulse, which further pulse rotates the spins of the protons of the tissue through 180°.

9. A method for determining a local property of a biological tissue, in which method, following excitation by an NMR sensor arranged at the distal end of a flexible shaft of an ablation catheter, adjacently to the point of the tissue to be measured, an NMR response signal of the tissue is generated and the local tissue property is determined on the basis of this NMR signal.

10. The method according to claim 9, wherein, prior to the generation of the NMR signal, the axis of an excitation cone of the NMR sensor is oriented substantially perpendicularly to the tissue surface.

11. The method according to claim 10, wherein the NMR sensor is oriented:
by actuating at least one pull cable fastened to the NMR sensor, such that a pivoting and/or rotation of the NMR sensor is brought about, and/or
by rotating the shaft.

12. The method according to claim 9, wherein the distal end of the shaft is displaced in the direction of the longitudinal axis of the shaft in such a way that the distal end of the shaft bears against the surface of the tissue to be measured.

13. The method according to claim 9, wherein intermittently between the determination of the local tissue property on the basis of the NMR signal, a shaft tip arranged at the distal end of the shaft is supplied with a current or a voltage is applied to the shaft tip.

14. A computer program product for determining a local property of a biological tissue, said computer program product comprising program code means for executing a computer program following implementation thereof in a data processing device, wherein the program code means are intended to execute the method according to claim 9 following the implementation in the data processing device.
